(19) United States
(12) Patent Application Publication
(10) Pub. No.: US 2021/0145087 A1
(43) Pub. Date: May 20, 2021

(54) Mask Pack Supporting Band With Face Lifting Function

(71) Applicant: Sang Yeon Park, Seoul (KR)

(72) Inventor: Sang Yeon Park, Seoul (KR)

(21) Appl. No.: 16/488,814

(22) PCT Filed: Feb. 19, 2018
(86) PCT No.: PCT/KR2018/002011

§ 371 Date: Aug. 26, 2019

(30) Foreign Application Priority Data
Feb. 27, 2017 (KR) 10-2017-0025272
Jan. 22, 2018 (KR) 10-2018-0007561

(51) Int. Cl. A41D 13/11

(52) U.S. Cl. CPC A41D 13/11

(57) ABSTRACT

Disclosed is a mask pack supporting band which lifts the entirety of a user's jaw and face with an appropriate tensile force in a state in which a mask pack is applied to the face, so as to prevent skin laxity and aging together with provision of a skin care function, and thus to freshen and tighten the facial skin. A supporting band is formed of an elastic material, such as a synthetic resin, to have a semicircular shape so as to be fixed to the user's head, fixing clips to fix the pulled mask pack are installed on the outer surface of the supporting band, binding straps to fix the supporting band worn on the head are provided at the upper end of the supporting band, a plurality of hanging protrusions is formed at both ends of a semicircular support unit, a plurality of pulling straps is installed at the upper end of the mask pack, used together with the supporting band, at equal intervals so as to extend farther outward than a general mask pack, the pulling straps of the mask pack attached to the user's face are pulled up and fixed to the fixing clips of the supporting band, and thereby, the user's facial skin, to which the mask pack is attached, may be lifted up.

### TECHNICAL FIELD

The present invention relates to a mask pack supporting band which provides a face and jaw lifting function together with a skin care function when a mask pack for facial skin care is used, so as to prevent skin laxity and aging and thus to freshen and tighten the facial skin.

### BACKGROUND ART

In order to maintain a youthful and healthy appearance, interest in plastic surgery, exercise, skin care, etc. is rising, and thus, as one method of facial skin care, demand for mask packs is currently increasing.

A term ‘pack’ originally has the meaning of surrounding, and a mask pack is a kind of functional cosmetic which surrounds the face with a skin cosmetic material, removes contaminants from pores of the skin and effectively provides moisture and cosmetic ingredients to the skin, so as to stimulate blood circulation and thus to restore a skin physiological function.

Mask packs may effectively provide various nutritional components to the skin, and thus provide various effects, such as moisturizing, smoothing, exfoliation and elimination of waste, skin restoration, improvement in skin elasticity, whitening, etc., due to these skin cosmetic components.

The mask pack used for skin care may generally have a structure in which a skin care cosmetic composition is adsorbed onto one of various materials, such as a non-woven fabric sheet, a knitted fabric sheet or a cotton fabric sheet, and be firmly attached to the face.

Since the mask pack in which the skin care cosmetic composition is adsorbed onto the material is merely attached to the face, a user needs to lay still while using the mask pack, and thus experiences inconvenience.

Particularly, the mask pack causes skin care cosmetic components to be adsorbed onto the face and may thus provide skin care effects, but is insufficient to expect functions of tightening the skin of the face or neck with an appropriate tensile force to impart elasticity to the skin that has lost its elasticity and to prevent the skin from sagging.

In order to overcome these drawbacks, mask packs which serve to tighten the face while mask-packing are provided.

As related art documents aiming to tighten the face while mask-packing, there are Korean Patent Registration No. 10-1166153 entitled “Mask beauty pack” (Patent Document 1) and Korean Patent Unexamined Publication No. 10-2016-0144044 entitled “Mask pack with cross coupling arm means (Patent Document 2).

Patent Document 1 discloses a mask pack of a sheet shape for cosmetic effects of the face and the jaw, and the mask pack may be firmly attached to the face in a stable and elastic manner while sufficiently pulling the jaw using ear loops alone.

Further, Patent Document 2 discloses a mask pack with arms intersecting at the jaw to surround the neck, in which a fold cut line is formed at the jaw of a face main body of the mask pack, first and second arms are provided at both sides of the fold cut line, the first arm and the second arm are spaced apart from each other by the distance of a spacing groove, both arms are pulled to intersect each other and thus overlap on the fold cut line so as to increase fixing power of the jaw when the mask pack is used, split cut lines are provided at regions of the face main body outside the upper ends of both arms, and both arms, which intersect each other, are inserted into the split cut lines so that the adhesive force of the mask pack to the face and the neck is maintained due to an increase in frictional force with the face main body, even if there is no fixing unit.

The mask packs disclosed in Patent Document 1 and Patent Document 2, which include units to pull up the jaw part, pull up the jaw part while the mask packs are used, and thus provide proper elasticity to the skin of the jaw part.

Therefore, as compared to conventionally used general mask packs, these mask packs not only provide original skin care functions but also pull up the jaw part, but, in the case of the mask pack of Patent Document 1, the jaw is pulled up using the ear loops extending from the mask pack as fixing units hanging on user's ears, and thus, a proper product needs to be appropriately selected depending on the user's face size, the user feels pressure when the fixed state of the ear loops hanging on the ears is maintained for a long time, and a jaw pulling effect is insufficient.

Further, in the case of the mask pack of Patent Document 2, the arms extending from the lower end of the mask pack intersect each other and are pulled up so as to provide tensile force to the jaw part, but the arms are not sufficiently capable of effectively pulling up the user's jaw part to provide tensile force.

Particularly, the mask packs of Patent Document 1 and Patent Document 2 mainly pull up the jaw part to provide elasticity to the skin of the jaw part but include no unit to pull up the face part to provide elasticity to the facial skin, and thus, it is difficult to expect a facial skin lifting effect.

### DISCLOSURE

### Technical Problem

An object of the present invention devised to solve the problem lies on a mask pack supporting band which may lift the entirety of the face and the jaw with an appropriate tensile force, in the state in which a mask pack for skin care is attached to the face, so as not only to provide skin care effects but also to prevent skin laxity and aging, and thus to freshen and tighten the facial skin.

### Technical Solution

The object of the present invention can be achieved by providing a mask pack supporting band formed of an elastic material, such as a synthetic resin, to have a semicircular shape so as to be fixed to a user's head, in which fixing clips to fix the pulled mask pack are installed on the outer surface of the supporting band, binding units to fix the supporting band worn on the head are provided at the upper end of the supporting band, a plurality of hanging protrusions are formed at both ends of a semicircular support unit, pulling straps are installed at the upper end of the mask pack, used together with the supporting band, at equal intervals so as to extend farther outward than a general mask pack, the pulling straps of the mask pack attached to the user's face are pulled up and fixed to the fixing clips of the supporting band, and thereby, the user's facial skin is lifted up in the state in which the user wears the mask pack on the face.

### Advantageous Effects

A mask pack supporting band in accordance with the present invention is formed of an elastic material, such as a synthetic resin, to have a semicircular shape so as to be fixed to a user's head, includes fixing clips installed on the outer surface of the supporting band to fix the pulled mask pack, binding units provided at the upper end of the supporting band to fix the supporting band worn on the head, and a plurality of hanging protrusions formed at both ends of a semicircular support unit, and lifts up the entirety of the user's face, to which the mask pack is attached, with an appropriate tensile force by pulling up pulling straps, installed at the upper end of the mask pack, used together with the supporting band, at equal intervals so as to extend farther outward than a general mask pack, and fixing the pulling straps to the fixing clips of the supporting band, and thereby provides facial skin care effects and prevents skin laxity and aging, thus keeping the skin fresh and elastic.

### DESCRIPTION OF DRAWINGS

FIG. 1 is a perspective view of a supporting band in accordance with the present invention.

FIG. 2 is a side view of the supporting band in accordance with the present invention.

FIG. 3 is a plan view of the supporting band in accordance with the present invention.

FIG. 4 is a cross-sectional view of a portion of the supporting band in accordance with the present invention.

FIG. 5 is a perspective view of a jaw lifting band in accordance with the present invention.

FIG. 6 is an exemplary view of a mask pack used in the present invention.

FIG. 7 is an exemplary view of a jaw mask pack used in the present invention.

FIG. 8 is a perspective view of a supporting band in accordance with another embodiment of the present invention.

FIG. 9 is a perspective view of a binding support unit of the supporting band of FIG. 8.

FIG. 10 is a perspective view of the binding support unit of FIG. 9, as seen from a different direction.

FIG. 11 is a perspective view of a semicircular support unit of the supporting band of FIG. 8.

FIG. 12 is a plan view of the semicircular support unit of FIG. 11.

FIG. 13 is a perspective view of an upper support unit of the supporting band of FIG. 8.

FIG. 14 is an exploded perspective view illustrating the combination of the binding support unit, the semicircular support unit and the upper support unit applied to the supporting band of FIG. 8.

FIG. 15 is a cross-sectional view of an upper coupler of the binding support unit in accordance with the present invention.

FIG. 16 is a cross-sectional view of a front coupler of the binding support unit in accordance with the present invention.

FIG. 17 is an exemplary perspective view of a supporting band in accordance with yet another embodiment of the present invention.

FIG. 18 is a perspective view of a fixture to fix a mask pack used in the present invention.

<Description of numerals and marks>

| | |
|---|---|
| (10)-supporting band | (20)-semicircular support unit |
| (21)-upper hanging loop | (22)-lower hanging loop |
| (23)-insertion part | (24)-hanging protrusion |
| (25)-prominence and depression part | (30)-fixing clip |
| (40)-binding strap | (41)-Velcro tape |
| (50)-jaw lifting band | (51)-supporting part |
| (52)-hanging piece | (53)-hanging hole |
| (56)-protruding thick part | (60)-mask pack |
| (61)-pulling strap | (62)-dotted line |
| (70)-jaw mask pack | (71)-pulling strap |
| (100)-semicircular support unit | (110)-length adjustment piece |
| (111)-prominence and depression part | (120)-fixing clip |
| (121)-protrusion | (200)-upper support unit |
| (210)-length adjustment piece | (211)-prominence and depression part |
| (212)-protrusion | (300)-binding support unit |
| (310)-main body | (320)-hanging loop |
| (321)-insertion part | (323)-binding hole |
| (324)-binding string | (326)-hanging rod |
| (327)-fixing clip | (330)-upper coupler |
| (331)-hanging piece | (332)-upper coupling groove |
| (333)-hanging protrusion | (340)-front coupler |
| (341)-hanging piece | (342)-front coupling groove |
| (343)-hanging protrusion | (344)-coupling hole |
| (345)-hanging protrusion | (346)-adjustment bolt |
| (350)-coupling part | (351) screw hole |
| (352)-hanging protrusion | (380)-mask pack fixture |
| (381)-fixing clip | |

### BEST MODE

Reference will now be made in detail to the preferred embodiments of the present invention, examples of which are illustrated in the accompanying drawings.

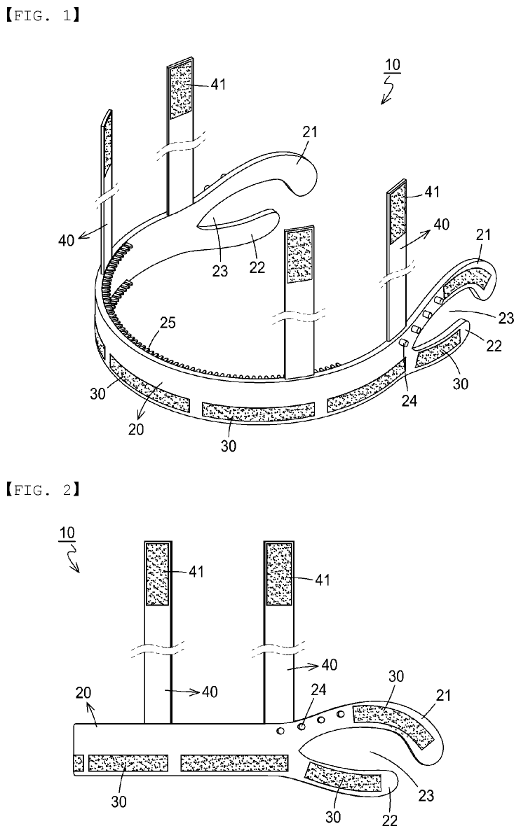

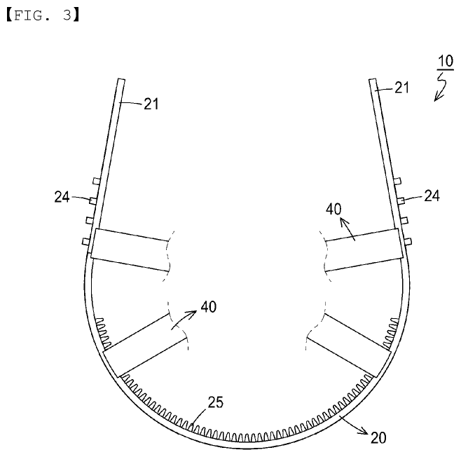

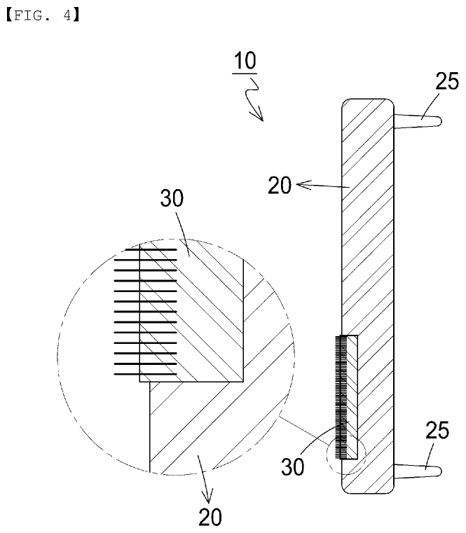

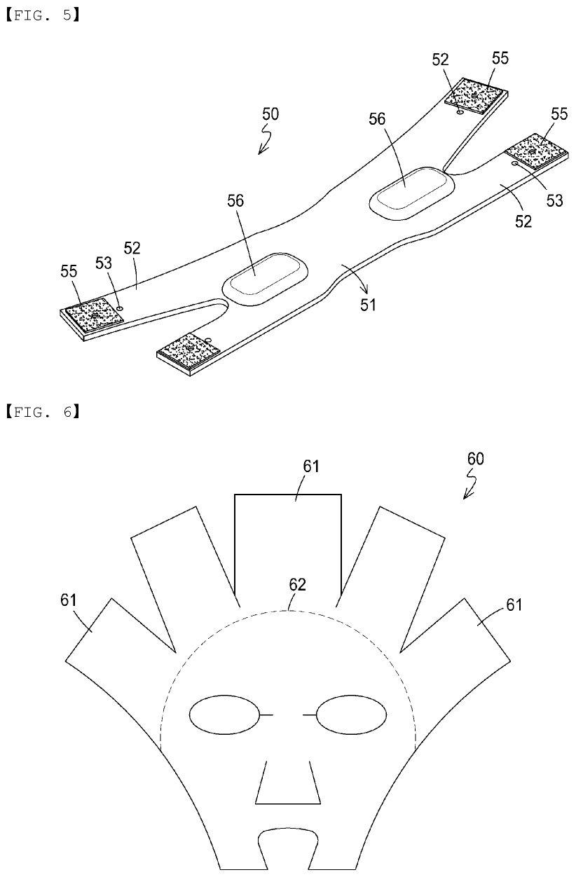

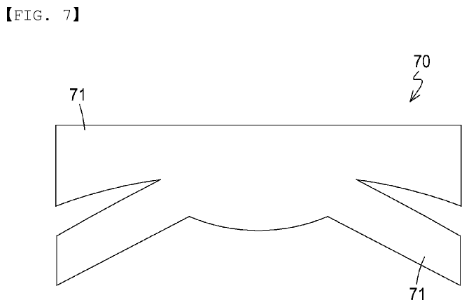

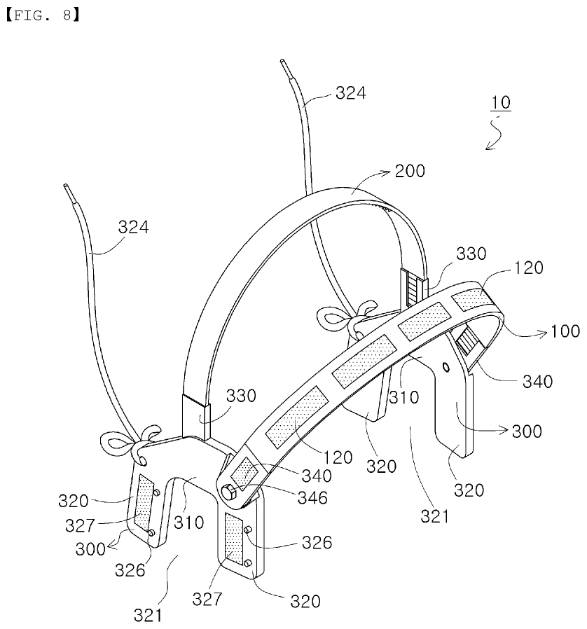

FIGS. 1 to 4 are views illustrating the configuration of a supporting band 10 in accordance with the present invention which a user wears on his/her head so that a mask pack 60 is bonded to the supporting band 10 with an appropriate tensile force so as to lift the user's facial skin, FIG. 5 is view illustrating the configuration of a jaw lifting band 50 functioning to lift the jaw, and FIGS. 6 and 7 are exemplary views of the mask pack 60 used in the present invention.

The supporting band 10 to bond and fix the mask pack 60 under tension is configured, as follows.

A semicircular support unit 20 is formed of an elastic material, and the semicircular support unit 20 is formed in the shape of a semicircular headband having a width of about 4-5 cm and a thickness of about 0.2-0.5 cm so as to be comfortably worn on the head.

The width and thickness of the semicircular support unit 20 are not limited.

An upper hanging loop 21 and a lower hanging loop 22 are provided at both ends of the semicircular support unit 20 so as to form an insertion part 23 therebetween and thus, when the supporting band 10 is worn on the head, the insertion part 23 is located at a user's ear, and a plurality of hanging protrusions 24 arranged at equal intervals is formed at both ends of the semicircular support unit 20.

Fixing clips 30 arranged at equal intervals are integrally formed at the edge of the outer surface of the semicircular support unit 20, and the fixing clips 30 may include small and fine needles, and may be bonded to the semicircular support unit 20 or be formed through insert injection molding.

In addition to needles, any member to which the mask pack is bonded and fixed, such as a Velcro tape, a hanging loop or the like, may be applied as the fixing clips 30.

Further, a prominence and depression part 25 is formed on the inner surface of the semicircular support unit 20, and thus, when the supporting band 10 is worn on the head, the prominence and depression part 25 engages with the hair and thus the semicircular support unit 20 is stably fixed to the hair without slipping.

Binding straps 40 are installed at the upper portion of the semicircular support unit 20 constituting the supporting band 10, and Velcro tapes 41 are attached to ends of the binding straps 40 so that the supporting band 10 is mounted on the user's head and then fixed using the binding straps 40, thereby firmly fixing the supporting band 10.

Here, the semicircular support unit 20 constituting the supporting band 10 is not limited to the semicircular shape, and may be manufactured to have a circular shape.

Together with the supporting band 10, in order to effectively lift the user's jaw, the jaw lifting band 50 may be used.

The jaw lifting band 50 is formed of a material having excellent elasticity, such as silicone resin, rubber or an elastic band, and includes a supporting part 51 having a width of 5-10 cm, a length of 30-40 cm and a thickness of 0.2-0.5 cm, and protruding thick parts 56 and hanging pieces 52, each of which includes hanging holes 53 and a Velcro tape 55, formed at both sides of the supporting part 51.

The hanging holes 53 are provided in a plural number so to as adjust a degree of lifting of the jaw lifting band 50 through length adjustment, when the jaw lifting band 50 is fixed to the supporting band 10 by inserting the hanging protrusions 24 into the hanging holes 53.

The mask pack 60 used together with the supporting band 10 may be configured, on the assumption that the size of a generally used mask pack is indicated by the position of a dotted line 62 shown in FIG. 6, such that a plurality of pulling straps 61 extends upwards from the position of the dotted line 62 so as to be easily bonded to fixing clips 30 of the supporting band 10 using the pulling straps 61.

Further, a jaw mask pack 70 used on the jaw may be configured such that pulling straps 71 extend from both ends of the jaw mask pack 70 so as to be stably bonded to the fixing clips 30 located at both ends of the supporting band 10.

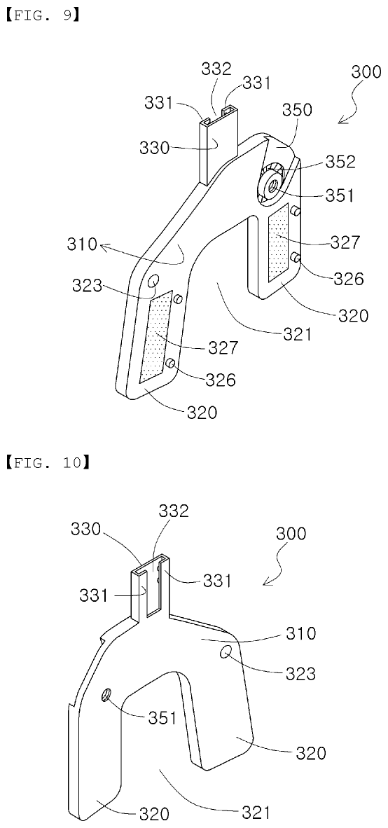

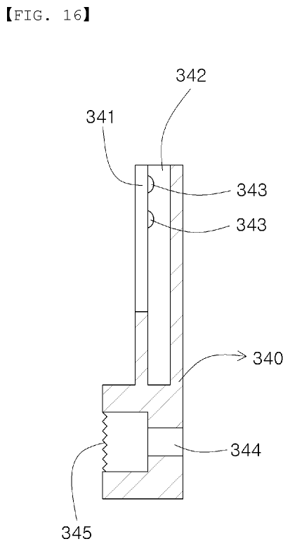

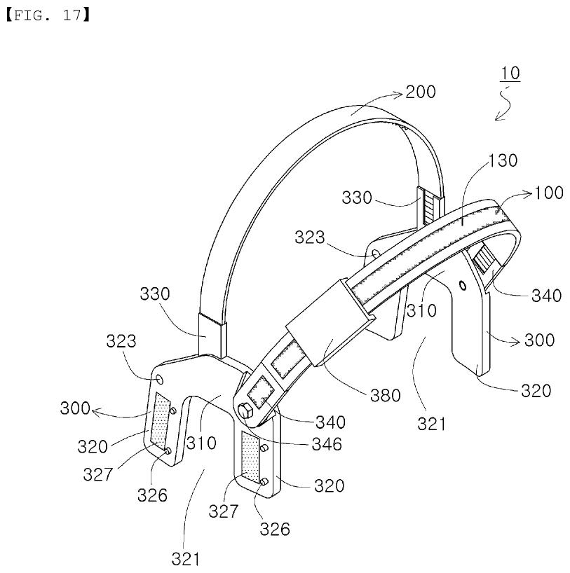

Another embodiment of the present invention will be described below with reference to FIGS. 8 to 16.

That is, as shown in FIG. 8, a supporting band 10 includes a semicircular support unit 100, an upper support unit 200, and binding support units 300 located at both sides of the supporting band 10, the semicircular support unit 100 and the upper support unit 200 are assembled with the binding support units 300 such that the lengths of the semicircular support unit 100 and the upper support unit 200 are adjustable and the angle of the semicircular support unit 100 is adjustable.

Hereinafter, the configurations of the semicircular support unit 100, the upper support unit 200 and the binding support units 300 constituting the supporting band 10 will be described.

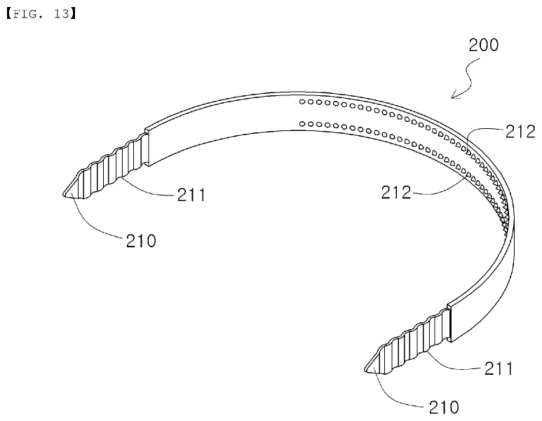

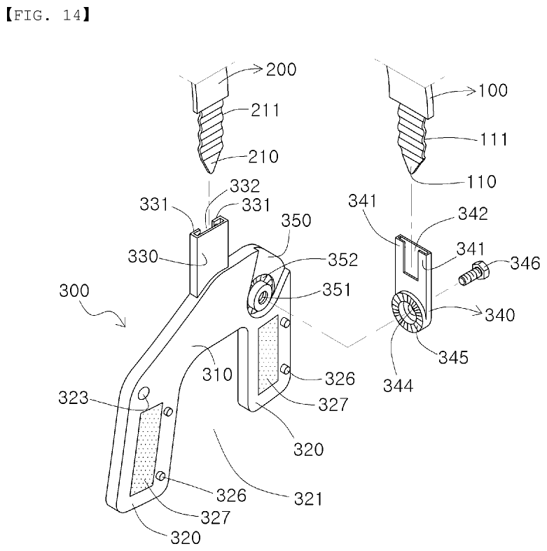

The semicircular support unit 100 is formed of an elastic material, such as a synthetic resin, to have a semicircular shape through injection molding, length adjustment pieces 110 provided with prominence and depression parts 111 are formed at both ends of the semicircular support unit 100, and the length adjustment pieces 110 are formed of a metal plate or a resin plate which does not rust and has elasticity, as shown in FIGS. 11 to 12.

Fixing clips 120 arranged at equal intervals are provided on the front surface of the semicircular support unit 100, and the fixing clips 120 include small and fine needles, and are bonded to the front surface of the semicircular support unit 100 or are formed through insert injection molding.

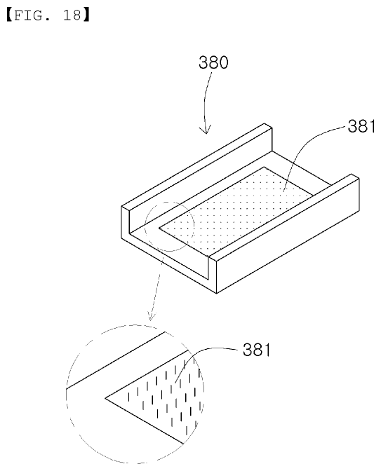

In the configuration of the semicircular support unit 100, no fixing clips 120 may be provided on the front surface of the semicircular support unit 100, and a Velcro tape 130 may be bonded thereto and a separate mask pack fixture 380 may be coupled to the semicircular support unit 100 so as to firmly fix the mask pack, as shown in FIG. 17.

Here, a fixing clip 381 including fine needles is provided on the inner surface of the mask pack fixture 380 and thus firmly fix the mask pack located on the front surface of the semicircular support unit 100, as shown in FIG. 18.

Further, protrusions 121 are provided on the rear surface of the semicircular support unit 100, and thus, when the supporting band 10 is worn on the head, the protrusions 121 engage with the hair and thus the semicircular support unit 100 is stably fixed to the hair without slipping.

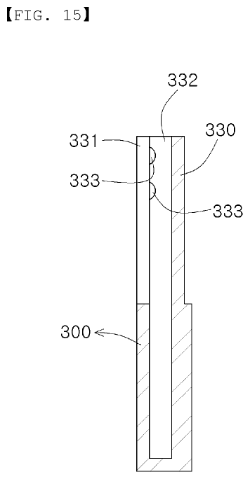

The upper support unit 200 is formed of an elastic material, such as a synthetic resin, to have a semicircular shape through injection molding, length adjustment pieces 210 provided with prominence and depression parts 211 are formed at both ends of the upper support unit 200, and the length adjustment pieces 210 are formed of a metal plate or a resin plate which does not rust and has elasticity, as shown in FIG. 13.

Protrusions 212 are provided at both edges of the rear surface of the upper support unit 200, and thus, when the supporting band 10 is worn on the head, the protrusions 212 engage with the hair and thus the upper support unit 200 is stably fixed to the hair without slipping.

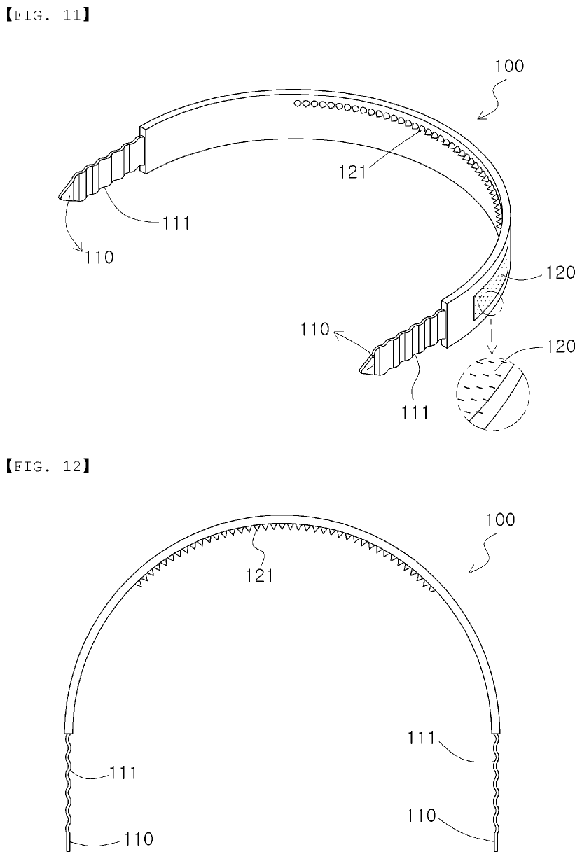

The binding support units 300 are symmetrically provided at both sides of the supporting band 10 and formed of a synthetic resin through injection molding, and an insertion part 321 is formed between both hanging loops 320 of a main body 310 constituting the binding support unit 300, as shown in FIGS. 9 and 10.

A fixing clip 327 including fine needles, and hanging rods 326 are provided on the outer surface of each hanging loop 320.

An upper coupler 330 is provided at the center of the upper end of the main body 310 of the binding support unit 300, the upper coupler 330 includes both hanging pieces 331 and an upper coupling groove 332, and hanging protrusions 333 are formed on the inner surfaces of the hanging pieces 331.

A coupling part 350 is provided at one side of the main body 310 of the binding support unit 300, and a screw hole 351 and a hanging protrusion 352 are formed at the center of the coupling part 350.

A front coupler 340 is rotatably coupled to the coupling part 350, and, here, the front coupler 340 is assembled to the coupling part 350 formed on the binding support unit 300 so that the angle of the front coupler 340 is adjustable, by matching a coupling hole 344 and a hanging protrusion 345 provided at the lower end of the front coupler 340 with the screw hole 351 and the hanging protrusion 351 of the coupling part 350 and then inserting an adjustment bolt 346 into the coupling hole 344 to be screwed to the screw hole 351.

A front coupling groove 342 is formed at the upper end of the front coupler 340 by hanging pieces 341, and hanging protrusions 343 are formed on the inner surfaces of the hanging pieces 341.

A binding hole 323 is formed at the rear end of the binding support unit 300, and a binding string 324 is connected to the binding hole 33.

The semicircular support unit 300 and the upper support unit 200 having the above-described configurations are assembled to both binding support units 300.

The assembly of the semicircular support unit 100 will be described below.

That is, when the length adjustment pieces 110 provided at both ends of the semicircular support unit 100 are inserted into the front coupling grooves 342 of the front couplers 340, each of which is provided at one side of the corresponding binding support unit 300, the prominence and depression parts 111 formed at the length adjustment pieces 110 contact and are locked with the hanging protrusions 343 provided on the inner surfaces of the hanging pieces 341, and thus, the length of the semicircular support unit 100 is adjusted depending on a degree of insertion of the length adjustment pieces 110 of the semicircular support unit 100 into the front couplers 340.

In order to adjust the angle of the semicircular support unit 100 inserted into the front couplers 340, when the adjustment bolts 346 coupled to the screw holes 351 of the coupling pars 350 are loosened, the front couplers 340 are rotated at a desired angle, and then the adjustment bolts 346 are retightened, adjustment of the angle of the semicircular support unit 100 coupled to the front couplers 340 is completed.

Next, the assembly of the upper support unit 200 will be described below.

That is, when the length adjustment pieces 210 provided at both ends of the upper support unit 200 are inserted into the upper coupling grooves 332 of the front couplers 330, constituting the binding support units 300, the prominence and depression parts 111 formed at the length adjustment pieces 210 contact and are locked with the hanging protrusions 333 provided on the inner surfaces of the hanging pieces 331, and thus, the length of the upper support unit 200 is adjusted depending on a degree of insertion of the length adjustment pieces 210 of the upper support unit 200 into the upper couplers 330.

Further, the binding strings 324 are connected to the binding holes 323 of the binding support units 300, and when the supporting band 10 is used, the binding strings 324 are tied at the rear portion of the user's head, thereby allowing the supporting band 10 to be stably fixed.

The supporting band 10 in accordance with the present invention is used while a mask pack for skin care is used, lifts the facial skin with an appropriate tensile force together with the effects of skin cosmetic components adsorbed onto the mask pack, and thus prevents skin laxity and aging, thereby aiding in keeping the skin fresh and elastic.

That is, when the supporting band 10 is worn on a user's head, the semicircular support unit 20 is located at a head part above the forehead and the upper and lower hanging loops 21 and 22 provided at both ends of the supporting band are located below and above the ears, and when the binding straps 40 located at the upper portion of the supporting band 10 are bound at the top of the head using the Velcro tapes 41, the supporting band 10 is firmly fixed to the user's head.

In this state, when the mask pack 60 onto which a skin care cosmetic composition is adsorbed is attached to the user's entire face, and then the pulling straps 61 located at the upper portion of the mask pack 60 are pulled up with an appropriate tensile force and contact the fixing clips 300 located on the outer surface of the semicircular support unit 20, the pulling straps 61 are bonded and fixed to the fixing clips 30.

Here, the fixing clips 30 include a plurality of thin and sharp needles, and thus, the pulling straps 61 of the mask pack formed of a non-woven fabric, a knitted fabric or a cotton fabric are easily bonded and fixed thereto.

When the mask pack 60 is attached to the user's entire face, and then the pulling straps 61 are pulled up and bonded and fixed to the fixing clips 30 of the supporting band 10, the facial skin is lifted up by the adhesive force of the mask pack 60, and thus, skin laxity and aging are prevented and the skin may be effectively kept youthful and tight.

Further, in the state in which the mask pack 60 is attached to the user's face, the state of the mask pack 60, which is firmly bonded and fixed to the fixing clips 30 of the supporting band 10 using the pulling straps 61, is maintained, and thus, the user may conveniently move freely without restriction, even while wearing the mask pack 60 on the face.

Further, in order to prevent skin laxity and aging of the jaw, the jaw mask pack 70 is separately used, and, when the jaw mask pack 70 configured as shown in FIG. 7 is located under the jaw and then the pulling straps 71 provided at both ends of the jaw mask pack 70 are pulled up and bonded and fixed to the fixing clips 30 of the supporting band 10, the saggy skin of the jaw is lifted up and thus proper elasticity is provided thereto.

Here, the skin laxity and aging of the jaw may be prevented using the jaw lifting band 50 configured as shown in FIG. 5.

That is, in the state in which the jaw mask pack 70 is attached to the user's jaw, when the supporting part 51 of the jaw lifting band 50 is located under the jaw, and then the hanging pieces 52 formed at both sides of the supporting part 51 are pulled up and the hanging holes 53 formed at the hanging pieces 52 are hung on the hanging protrusions 24 formed on the supporting band 10, the skin of the jaw is lifted up by elasticity of the jaw lifting band 50, and thus, skin laxity and aging are prevented, and thereby skin care effects in which the skin is kept youthful and tight may be expected.

Here, the protruding thick parts 56 protruding from the jaw lifting band 50 press the saggy skin at both sides of the jaw while pulling the skin under tension, and thus effective skin care may be expected.

Further, the supporting band 10 shown in FIGS. 8 to 16 is not greatly different in use from the above-described supporting band 10.

That is, when the supporting band 10 is worn on a user's head, the semicircular support unit 100 is located at a head part above the forehead, the binding support units 300 provided at both ends of the supporting band 10 are located at the ears, the upper support unit 200 is located at the top of the head, and when the binding strings 324 are tied at the rear portion of the user's head, the supporting band 10 is firmly fixed to the user's head.

Here, the protrusions 121 and 212 provided on the rear surfaces of the semicircular support unit 100 and the upper support unit 200 engage with the hair, and thus, firmer fixation of the supporting band is carried out.

Further, in order to adjust the lengths of the semicircular support unit 100 and the upper support unit 200 during use, the user may simply adjust the lengths of the semicircular support unit 100 and the upper support unit 200 through a process of separating and reinserting the length adjustment pieces 110 and 210 from and into the front coupling grooves 342 and the upper coupling grooves 332 of the front couplers 340 and the upper couplers 330.

Since, in this process, the prominence and depression parts 111 and 211 formed on the length adjustment pieces 110 and 210 elastically contact the hanging protrusions 343 and 333 of the hanging pieces 341 and 331, when the lengths of the length adjustment pieces 110 and 210 are properly adjusted, the adjusted states thereof are maintained unless external force is applied thereto.

In order to adjust the angle of the semicircular support unit 100, the adjustment bolts 346 located at the front couplers 340 are loosened, the front couplers 340 are rotated at a desired angle, and then the adjustment bolts 346 are retightened and then fixed.

Here, the hanging protrusions 352 and 345 provided at the coupling parts 350 of the binding support units 300 and the front couplers 340 are engaged with each other, and thus when the adjustment bolts 346 are tightened after adjustment of the angle, the position of the semicircular support unit 100 is firmly fixed at the set angle.

In the state in which the supporting band 10 is worn on the user's head, when the mask pack 60 onto which a skin care cosmetic composition is adsorbed is attached to the user's entire face and then the upper portion of the mask pack 60 is pulled up with an appropriate tensile force and contacts the fixing clips 120 located on the outer surface of the semicircular support unit 100, the upper end of the mask pack 60 is bonded and fixed to the fixing clips 120.

When the mask pack 60 is attached to the user's entire face, and then the upper end of the mask pack 60 is pulled up and fixed to the fixing clips 120 of the supporting band 10, the facial skin is lifted up by the adhesive force of the mask pack 60, and thus, skin laxity and aging are prevented and the skin may be effectively kept youthful and tight.

Further, in the state in which the mask pack 60 is attached to the user's entire face, the state in which the upper end of the mask pack 60 is firmly bonded and fixed to the fixing clips 120 of the supporting band 10 is maintained, and thus, the user may conveniently move freely without restriction, even while wearing the mask pack 60.

In order to achieve firmer bonding in the state in which the upper end of the mask pack 60 is bonded and fixed to the fixing clips 120 of the semicircular support unit 100, the mask pack fixtures 380 shown in FIG. 18 are coupled to the semicircular support unit 100 at equal intervals, and thereby, firmer bonding may be expected.

In the configuration of the supporting band 10 in accordance with the present invention, the Velcro tape 130 may be provided on the front surface of the semicircular support unit 100 and then the mask pack 60 may be firmly fixed to the semicircular support unit 100 using the mask pack fixture 380, as shown in FIG. 17.

That is, after the supporting band 10 is worn on the user's head, when the upper end of the mask pack 60 attached to the user's face is pulled up and is then located on the front surface of the semicircular support unit 100, the upper end of the mask pack 60 is primarily bonded to the Velcro tape 130, the mask pack fixture 380 is coupled to the semicircular support unit 100 in this state so that the mask pack 60 is fixed to the front surface of the semicircular support unit 100, and in this case, the fixing clip 381 including fine needles is formed on the inner surface of the mask pack fixture 380 and thus serves to firmly fix the mask pack 60.

Further, in order to prevent skin laxity and aging of the jaw, the jaw mask pack 70 is separately used, and, when the jaw mask pack 70 configured as shown in FIG. 7 is located under the jaw and then the pulling straps 71 provided at both ends of the jaw mask pack 70 are pulled up and fixed to the fixing clips 327 or the hanging rods 326 of the binding support units 300, the saggy skin of the jaw is lifted up, and thus proper elasticity is provided thereto.

1. (canceled)

2. A mask pack supporting band with a face lifting function in which upper couplers (330), each comprising hanging pieces (331) and an upper coupling groove (332), are provided at centers of upper ends of both binding support units (300), each comprising hanging loops (320) and an insertion part (310), an upper support unit (200) is fixed to both the binding support units (300) by inserting length adjustment pieces (210) provided at both ends of the upper support unit (200) into the upper coupling grooves (332) of the upper couplers (330), coupling parts (350), each comprising a screw hole (351) and a hanging protrusion (352), are provided at one sides of the binding support units (300), front couplers (340), each comprising a hanging protrusion (345), are located at the coupling parts (350) so that the hanging protrusions (352) of the coupling parts (350) and the hanging protrusions (345) of the front couplers (345) are located side by side and are then coupled using adjustment bolts (346), hanging pieces (341) and a front coupling groove (342) are provided at each of upper ends of the front couplers (340), and a semicircular support unit (100) is fixed to the front couplers (340) by inserting length adjustment pieces (110) provided at both ends of the semicircular support unit (100) into the front coupling grooves (342).

3. The mask pack supporting band according to claim 2, wherein hanging protrusions (333) (343) are provided on the hanging pieces (331) (341) of the upper couplers (330) and the front couplers (340), prominence and depression parts (211) (111) are provided on the length adjustment pieces (210) (110) of the upper support unit (200) and the semicircular support unit (100), and lengths of the upper support unit (200) and the semicircular support unit (100) are adjustable by hanging the prominence and depression parts (211) (111) on the hanging protrusions (333) (343) of the upper couplers (330) and the front couplers (340).

4. The mask pack supporting band according to claim 2, wherein a mask pack fixture (380) is coupled to a front surface of the semicircular support unit (100) so as to fix a mask pack located on the front surface of the semicircular support unit (100).
